(19) United States
(12) Patent Application Publication
(10) Pub. No.: US 2026/0369677 A1
(43) Pub. Date: Nov. 19, 2026

(54) ADDITIVE FOR ELECTROLYTE OF LITHIUM ION BATTERY, ELECTROLYTE, AND LITHIUM ION BATTERY

(71) Applicant: MURATA MANUFACTURING CO., LTD., Kyoto (JP)

(72) Inventors: Xiaoyin XUE, Shanghai (CN); Fan YANG, Shanghai (CN); Cheng ZHU, Shanghai (CN); Kazumasa TAKESHI, Shanghai (CN)

(21) Appl. No.: 19/550,583

(22) Filed: Feb. 26, 2026

(30) Foreign Application Priority Data
Apr. 30, 2025 (CN) 2025105712886

(51) Int. Cl. H01M 10/0567; C07D 413/06; H01M 10/0525; H01M 10/0569

(52) U.S. Cl. CPC H01M 10/0567; C07D 413/06; H01M 10/0525; H01M 10/0569; H01M 2300/004

(57) ABSTRACT

An additive for an electrolyte of a lithium ion battery, an electrolyte and a lithium ion battery are provided. The additive includes a compound having a structure of formula (1), wherein R is a substituted or unsubstituted linear C1-6 hydrocarbon group. The additive for an electrolyte of a lithium ion battery, the electrolyte and the lithium ion battery of the present application can provide improved cycle performance and cycle impedance.

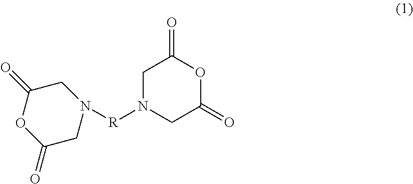

### CROSS REFERENCE TO RELATED APPLICATIONS

The present application claims priority to Chinese patent application no. 2025105712886, filed on Apr. 30, 2025, the entire contents of which are incorporated herein by reference.

### BACKGROUND

The present application relates to the field of lithium ion batteries, and in particular, to an additive for an electrolyte of a lithium ion battery, an electrolyte and a lithium ion battery.

In recent years, with the continuous development of electronic technology, demand for a battery apparatus supporting energy supply of electronic devices is increasing continuously. Today, batteries capable of storing more electricity and capable of outputting high power are needed. Conventional lead-acid batteries and nickel-hydrogen batteries, etc. cannot meet the requirements of new electronic products such as mobile devices, e.g. smart phones and fixed devices, e.g. power storage systems. Therefore, lithium batteries have attracted wide attention. In the development process of lithium ion batteries, the capacity and performance thereof have been improved effectively.

A lithium ion battery includes a positive electrode, a negative electrode, and an electrolyte. Electrolyte additives for lithium ion batteries have a significant effect on the overall performance of the batteries. Electrolyte additives for lithium ion batteries have been studied. The use of additives in electrolytes of lithium ion batteries may be beneficial in improving the capacity or cycle performance of the batteries, but may also lead to increase in impedance of the batteries.

Therefore, it is still necessary to develop an electrolyte additive, an electrolyte and a lithium ion battery with improved cycle performance and reduced cycle impedance.

### SUMMARY

The present application relates to providing, in an embodiment, an additive for an electrolyte of a lithium ion battery, an electrolyte and a lithium ion battery, so as to solve the problem of poor cycle performance and cycle impedance of lithium ion batteries in the related art.

In an embodiment, the present application provides an additive for an electrolyte of a lithium ion battery, including a compound having a structure of formula (1)

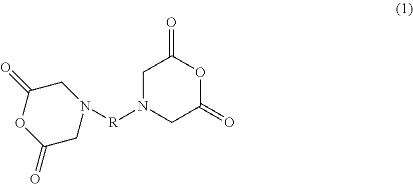

wherein R is a substituted or unsubstituted linear or branched C1-6 hydrocarbon group.

Further, in formula (1), R is an unsubstituted linear or branched C1-6 alkyl group.

Further, the compound having the structure of formula (1), in an embodiment, is methylenediaminetetraacetic dianhydride, ethylenediaminetetraacetic dianhydride, propylenediaminetetraacetic dianhydride, butylenediaminetetraacetic dianhydride, pentanediaminetetraacetic dianhydride, hexamethylenediaminetetraacetic dianhydride, 4,4′-(propane-1,2-diyl)bis(morpholine-2,6-dione), 4,4′-(butane-1,2-diyl)bis(morpholine-2,6-dione), 4,4′-(pentane-1,2-diyl)bis(morpholine-2,6-dione), 4,4′-(hexane-1,2-diyl)bis(morpholine-2,6-dione), 4,4′-(butane-2,3-diyl)bis(morpholine-2,6-dione) or 4,4′-(hexane-3,4-diyl)bis(morpholine-2,6-dione).

Another aspect of the present application, in an embodiment, further provides an electrolyte of a lithium ion battery, including the additive of the described aspect, an organic solvent and a lithium salt.

Further, based on the total mass of the electrolyte, the amount of the additive is 0.005 wt %-0.5 wt % in an embodiment.

Further, the organic solvent includes a cyclic carbonate, a linear carbonate, or any combination thereof in an embodiment.

Further, the lithium salt is selected from a group consisting of lithium hexafluorophosphate, lithium bis(trifluoromethylsulfonyl)imide, lithium trifluoromethylsulfonate, lithium bis(fluorosulfonyl)imide, lithium tetrafluoroborate, lithium bis(oxalate) borate, lithium difluorooxalate borate, lithium perchlorate, or a combination thereof in an embodiment.

Still another aspect of the present application provides a lithium ion secondary battery, including: a positive electrode plate, a negative electrode plate, a separator, and the electrolyte in an embodiment.

Further, the positive electrode plate includes a ternary or lithium cobalt oxide positive electrode material in an embodiment.

Further, the negative electrode plate includes a graphite or silicon-carbon negative electrode material in an embodiment.

By applying the technical solution of the present application, in an embodiment, the compound of formula (1) is used as an electrolyte additive of a lithium ion battery, so that a positive electrode can be protected during battery cycling, thereby improving the first-cycle capacity and cycling discharge capacity of the battery, inhibiting increase of the impedance of the battery, and improving the discharge plateau. In the compound of formula (1), the anhydride structure can form a stable CEI film on the surface of the positive electrode, thereby eliminating the negative effect on the positive electrode caused by H₂O and HF generated by the electrolyte in the cycling process in an embodiment. As an electrolyte additive, in an embodiment, alkylenediaminetetraacetic dianhydride and derivatives thereof having a chain structure are added into an electrolyte, which can effectively inhibit increase in impedance of the battery, and the battery can exhibit better cycle performance, better cycle capacity and capacity retention rate, and exhibit a lower impedance after cycling, and after cycling, the battery can also retain high discharge capacity during high-rate discharge.

### BRIEF DESCRIPTION OF THE FIGURES

The drawings of the description, constituting a part of the present application, are used for providing further understanding of the present application, and the illustrative examples of the present application and illustrations thereof are used to explain the present application, but not inappropriate limitation on the present application. In the drawings:

FIG. 1 is a graph of cycle capacity of lithium ion batteries prepared using EDTAA as an electrolyte additive in the Examples and lithium ion batteries prepared using a basic electrolyte.

FIG. 2 shows charge and discharge curves of lithium ion batteries prepared using EDTAA as an electrolyte additive in the Examples and lithium ion batteries prepared using a basic electrolyte.

FIG. 3 is a graph of cycle impedance of lithium ion batteries prepared using EDTAA as an electrolyte additive in the Examples and lithium ion batteries prepared using a basic electrolyte.

FIG. 4 is a graph of capacity and temperature variations of lithium ion batteries prepared using EDTAA as an electrolyte additive in the Examples and lithium ion batteries prepared using a basic electrolyte.

FIG. 5 is a schematic diagram illustrating a mechanism of removal of H₂O and HF generated by an electrolyte in a cycling process by an additive compound according to the present application.

### DETAILED DESCRIPTION

It is to be noted that examples in the present application and features in the examples may be combined with one another in any suitable manner. Hereinafter, the present application will be described in further detail including with reference to the examples and figures according to an embodiment.
the present application provides an additive for an electrolyte of a lithium ion battery, including a compound having a structure of formula (1)

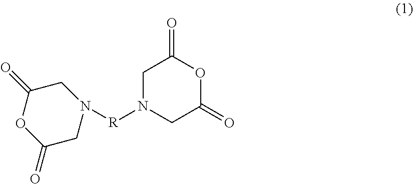

wherein R is a substituted or unsubstituted linear C1-6 hydrocarbon group.

The compound of formula (1) is used as an electrolyte additive of a lithium ion battery, so that a positive electrode can be protected during battery cycling, thereby improving the first-cycle capacity and cycling discharge capacity of the battery, inhibiting increase of the impedance of the battery, and improving the discharge plateau. In the compound of formula (1), the anhydride structure can form a stable CEI film on the surface of the positive electrode, thereby eliminating the negative effect on the positive electrode caused by H₂O and HF generated by the electrolyte in the cycling process. As an electrolyte additive, alkylenediaminetetraacetic dianhydride and derivatives thereof having a chain structure are added into an electrolyte, which can effectively inhibit increase in impedance of the battery, and the battery can exhibit better cycle performance, better cycle capacity and capacity retention rate, and exhibit a lower impedance after cycling, and after cycling, the battery can also retain high discharge capacity during high-rate discharge.

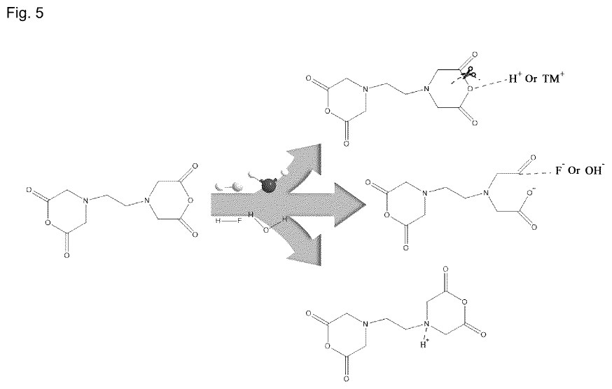

The mechanism of removal of H₂O and HF by the compound of formula (1) is as shown in FIG. 5 (taking ethylenediaminetetraacetic dianhydride as an example), the tetraacetic dianhydride moiety thereof can react with H₂O and HF to adsorb H₂O and HF generated by the electrolyte in the cycling process, thereby preventing the erosion of the battery electrolyte by H₂O, and reducing the degradation and decomposition of the electrolyte and the HF eventually formed. In addition, similar to the mechanism described above, the compound of formula (1) can be oxidized during the cycling and then undergo a ring-opening reaction, to form the CEI film on the surface of the positive electrode. The compound forms film on the surface of the electrode earlier than the electrolyte, preventing the electrolyte from continuous oxidation and reduction on the electrode surface, thereby achieving the effect of protecting the electrode during battery cycling, improving the first-cycle capacity and cycling discharge capacity of the battery, inhibiting increase of the impedance of the battery, and improving the discharge plateau. Therefore, the additive of the present application has the advantages of forming CEI and SEI films with lower impedance while ensuring the usability of the battery being unaffected, improving the cycle performance of the battery, prolonging the service life of the battery, etc.

The selection of the R group in the compound of formula (1) can affect performance thereof as an additive. If the carbon chain of the R group is too long, the molecules are too large when the CEI is formed on the surface of the positive electrode, thus the reaction sites are reduced, and the formed CEI film is not uniform enough, and it is unfavorable for intercalation and deintercalation of lithium ions.

In an embodiment, in formula (1), R is an unsubstituted linear or branched C1-6 alkyl group.

In an embodiment, the compound having the structure of formula (1) is methylenediaminetetraacetic dianhydride, ethylenediaminetetraacetic dianhydride, propylenediaminetetraacetic dianhydride, butylenediaminetetraacetic dianhydride, pentanediaminetetraacetic dianhydride, hexamethylenediaminetetraacetic dianhydride, 4,4′-(propane-1,2-diyl)bis(morpholine-2,6-dione), 4,4′-(butane-1,2-diyl)bis(morpholine-2,6-dione), 4,4′-(pentane-1,2-diyl)bis(morpholine-2,6-dione), 4,4′-(hexane-1,2-diyl)bis(morpholine-2,6-dione), 4,4′-(butane-2,3-diyl)bis(morpholine-2,6-dione) or 4,4′-(hexane-3,4-diyl)bis(morpholine-2,6-dione), more preferably, methylenediaminetetraacetic dianhydride, ethylenediaminetetraacetic dianhydride, propylenediaminetetraacetic dianhydride, 4,4′-(propane-1,2-diyl)bis(morpholine-2,6-dione), 4,4′-(butane-1,2-diyl)bis(morpholine-2,6-dione), and 4,4′-(butane-2,3-diyl)bis(morpholine-2,6-dione).

The chemical formulae of 4,4′-(propane-1,2-diyl)bis(morpholine-2,6-dione), 4,4′-(butane-1,2-diyl)bis(morpholine-2,6-dione), 4,4′-(pentane-1,2-diyl)bis(morpholine-2,6-dione), 4,4′-(hexane-1,2-diyl)bis(morpholine-2,6-dione), 4,4′-(butane-2,3-diyl)bis(morpholine-2,6-dione) and 4,4′-(hexane-3,4-diyl)bis(morpholine-2,6-dione) are sequentially:

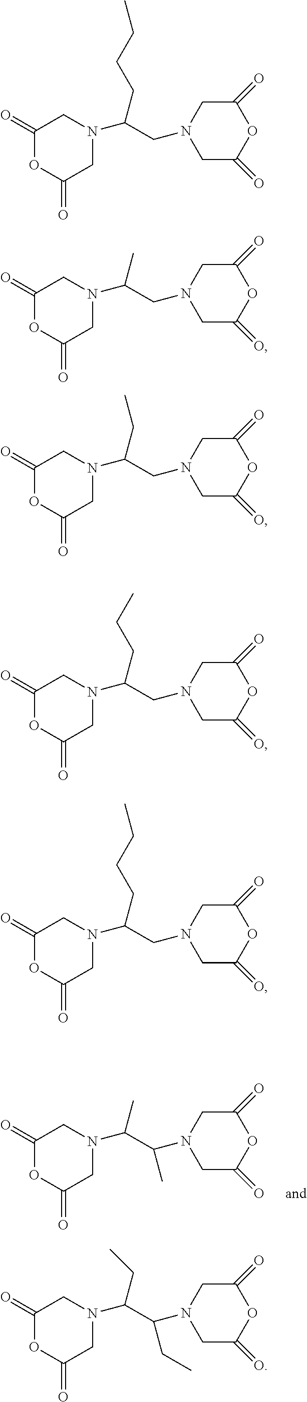

In an embodiment, the compound having the structure of formula (1) is ethylenediaminetetraacetic dianhydride, methylenediaminetetraacetic dianhydride, propylenediaminetetraacetic dianhydride, 4,4′-(propane-1,2-diyl)bis(morpholine-2,6-dione), 4,4′-(butane-1,2-diyl)bis(morpholine-2,6-dione), and 4,4′-(butane-2,3-diyl)bis(morpholine-2,6-dione).

The specific compounds described above are advantageous for further improving the cycle performance and reducing cycle impedance of lithium ion batteries according to an embodiment.

According to another aspect of the present application, provided is an electrolyte including an organic solvent, a lithium salt and the additive as described in the present application.

The electrolyte of the present application comprises the electrolyte additive as described in the present application, the compound of formula (1) is used as the electrolyte additive of the lithium ion battery, so that the positive electrode can be protected during battery cycling, thereby improving the first-cycle capacity and cycling discharge capacity of the battery, inhibiting increase of the impedance of the battery, and improving the discharge plateau. In the compound of formula (1), the anhydride structure can form a stable CEI film on the surface of the positive electrode, thereby eliminating the negative effect on the positive electrode caused by H₂O and HF generated by the electrolyte in the cycling process. As an electrolyte additive, alkylenediaminetetraacetic dianhydride and derivatives thereof having a chain structure are added into an electrolyte, which can effectively inhibit increase in impedance of the battery, and the battery can exhibit better cycle performance, better cycle capacity and capacity retention rate, and exhibit a lower impedance after cycling, and after cycling, the battery can also retain high discharge capacity during high-rate discharge.

In an embodiment, based on the total mass of the electrolyte, the amount of the additive is 0.005 wt %-0.5 wt %, or 0.005 wt %-0.4 wt %, or 0.005 wt %-0.3 wt %, or 0.005 wt %-0.2 wt %, or 0.005 wt %-0.1 wt %.

Using the additive content within the described range is beneficial to further improve the cycle performance and reduce the cycle impedance of the lithium ion battery.

In an embodiment, the organic solvent includes a cyclic carbonate, a linear carbonate, or any combination thereof.

In an embodiment, the organic solvent is selected from one or more of ethylene carbonate, propylene carbonate, butylene carbonate, fluoroethylene carbonate, dimethyl carbonate, diethyl carbonate, dipropyl carbonate, dihexyl carbonate, methylethyl carbonate, methylpropyl carbonate and propyl propionate. The list above only provides examples of the organic solvent used in the present application, but it is not limited thereto. A person skilled in the art would have been able to select a suitable organic solvent according to actual needs. In a preferred embodiment, at least one carbonate is used as the organic solvent of the electrolyte of the present application. In a preferred embodiment, at least one linear carbonate and at least one cyclic carbonate are used together as the organic solvent of the electrolyte of the present application.

In an embodiment, the lithium salt is selected from a group consisting of lithium hexafluorophosphate, lithium bis(trifluoromethylsulfonyl)imide, lithium trifluoromethylsulfonate, lithium bis(fluorosulfonyl)imide, lithium tetrafluoroborate, lithium bis(oxalate) borate, lithium difluorooxalate borate, lithium perchlorate, or a combination thereof.

When the electrolyte comprises a linear carbonate, a cyclic carbonate or any combination thereof, a lithium salt and the additive of the present application, it is beneficial to improve the cycle performance and reduce cycle impedance of the lithium ion secondary battery.

According to still another aspect of the present application, provided is a lithium ion secondary battery, including: a positive electrode plate, a negative electrode plate, a separator, and the electrolyte as described above.

In an embodiment, the positive electrode plate includes a ternary or lithium cobalt oxide positive electrode material.

In an embodiment, the negative electrode plate includes a graphite or silicon-carbon negative electrode material.

Hereinafter, the present application will be further described in further detail including in combination with specific examples according to an embodiment.

### EXAMPLES

### Preparation of Positive Electrode Plate

95.5 g of a positive electrode active material, lithium nickel cobalt aluminate NCA(LiNiCoAlO₂), 2.5 g of conductive carbon black, 1.9 g of polyvinylidene fluoride, and 0.1 g of a polyvinylpyrrolidone dispersant were mixed to obtain a positive electrode mixture, and the obtained mixture was dispersed in N-methylpyrrolidone to obtain a positive electrode mixture slurry. Then, the positive electrode mixture slurry was uniformly coated onto an aluminum foil to obtain a positive electrode current collector, the positive electrode current collector was dried, and a positive electrode plate was formed therefrom using a punch forming process.

### Preparation of Negative Electrode Plate

95.85 g of a mixture of silicon monoxide and graphite powder at a weight ratio of 9:1, 1 g of Super P, and 3.15 g of a binder, carboxy methyl cellulose (CMC) and styrene-butadiene latex (SBR) at a weight ratio of 1:1, and an appropriate amount of water were stirred to prepare a negative electrode slurry. Then, the obtained negative electrode slurry was uniformly coated on a copper foil to obtain a negative electrode current collector, the negative electrode current collector was dried, and a negative electrode plate was formed therefrom using a punch forming process.

### Preparation of Basic Electrolyte

20.75 g of ethylene carbonate, 56.75 g of dimethyl carbonate, 3.5 g of methylethyl carbonate, 0.4 g of lithium tetrafluoroborate and 18.6 g of lithium hexafluorophosphate were mixed to prepare a basic electrolyte, which were uniformly stirred for use.

### Preparation of Electrolytes of Examples 1-56

Electrolyte additives were added to the basic electrolyte and mixed uniformly for use, and the types and amounts of the additives are as shown in Table 1 below.

### Preparation of Electrolyte of Comparative Example 1

Same as the basic electrolyte, no additive was added.

### Battery Assembly

A CR2016 button battery was assembled in a dry laboratory. The positive electrode plate prepared by the steps above was used as a positive electrode, the negative electrode plate was used as a negative electrode, and the electrolytes prepared in the Examples and the Comparative Example were used as electrolytes. The positive electrode, the negative electrode, a separator and a battery housing of the button battery were assembled, and the electrolyte was injected. After the battery was assembled, the battery was allowed to stand for 12 hours for aging, thereby obtaining the button battery.

### Test of Battery Performance

The button batteries of the Examples and the Comparative Example were subjected to a first charge and discharge test and an impedance test at room temperature at a voltage of 2.0 V to 4.2 V. The button batteries of the Examples and the Comparative Example were subjected to a charge and discharge cycling test and an impedance test at room temperature at a voltage of 2.5 V to 4.2 V. The batteries in the Examples and Comparative Example above were first subjected to one cycle test of 0.1 C at 23 °C, and then subjected to a charge and discharge cycling test of 0.5 C charging and 5 C discharging at 60 °C for 100 times to determine the cycle retention rates of the batteries, and finally subjected to one charging test of 0.5 C at 60 °C to determine the impedance values of the batteries.

Capacity retention rate after 100 cycles [%]=(discharge capacity after 100th cycle/first discharge capacity (first cycle))×100%.

The cycle performance test results for the batteries of Examples 1-85 and Comparative Example 1 are shown in Table 1 below.

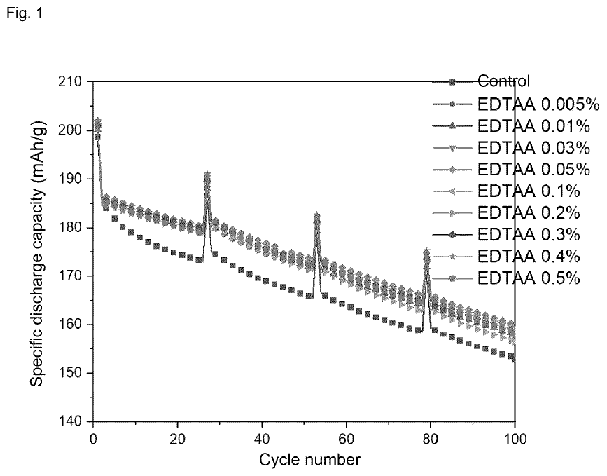

In addition, the cycle capacity, charge and discharge curve and cycle impedance of the lithium ion batteries prepared in Examples 1-9 and Comparative Example 1 are shown in FIGS. 1-3. In FIG. 1, the peaks in the curve are obtained by performing small-rate 0.1 C capacity recovery once after every 25 cycles.

TABLE 1

| | | | Cycle | First-cycle | 100th-cycle | Capacity |
|---|---|---|---|---|---|---|
| Example | | Addition | impedan | capacity | capacity | retention |
| No. | Additive | amount | ce (Ω) | (mAh/g) | (mAh/g) | rate |
| Comparative | Standard electrolyte without additive | — | 19.56 | 184.91 | 152.73 | 83.01% |
| Example 1 | | | | | | |
| 1 | Ethylenediaminetetraacetic dianhydride | 0.005% | 17.46 | 186.6 | 158.15 | 84.75% |
| 2 | Ethylenediaminetetraacetic dianhydride | 0.010% | 17.39 | 186.11 | 158.06 | 84.93% |
| 3 | Ethylenediaminetetraacetic dianhydride | 0.030% | 17.04 | 184.42 | 158.47 | 85.87% |
| 4 | Ethylenediaminetetraacetic dianhydride | 0.050% | 16.63 | 186.57 | 158.91 | 85.76% |
| 5 | Ethylenediaminetetraacetic dianhydride | 0.100% | 17.02 | 185.21 | 158.93 | 85.81% |
| 6 | Ethylenediaminetetraacetic dianhydride | 0.200% | 17.39 | 185.55 | 156.44 | 84.31% |
| 7 | Ethylenediaminetetraacetic dianhydride | 0.300% | 17.66 | 185.49 | 158.47 | 85.43% |
| 8 | Ethylenediaminetetraacetic dianhydride | 0.400% | 18.13 | 184.95 | 157.58 | 85.21% |
| 9 | Ethylenediaminetetraacetic dianhydride | 0.500% | 18.63 | 185.25 | 158.36 | 85.57% |
| 10 | Ethylenediaminetetraacetic dianhydride | 1% | 21.17 | 185.68 | 154.15 | 83.02% |
| 11 | Methylenediaminetetraacetic dianhydride | 0.005% | 17.17 | 186.42 | 158.18 | 84.85% |
| 12 | Methylenediaminetetraacetic dianhydride | 0.010% | 17.3 | 186.01 | 158.55 | 85.24% |
| 13 | Methylenediaminetetraacetic dianhydride | 0.030% | 17.2 | 184.28 | 159.03 | 86.30% |
| 14 | Methylenediaminetetraacetic dianhydride | 0.050% | 17.26 | 187.38 | 158.95 | 84.83% |
| 15 | Methylenediaminetetraacetic dianhydride | 0.100% | 17.6 | 186.17 | 159.06 | 85.44% |
| 16 | Methylenediaminetetraacetic dianhydride | 0.200% | 17.62 | 185.83 | 157.07 | 84.52% |
| 17 | Methylenediaminetetraacetic dianhydride | 0.300% | 17.76 | 185.17 | 157.51 | 85.06% |
| 18 | Methylenediaminetetraacetic dianhydride | 0.400% | 17.99 | 184.34 | 158.53 | 86.00% |
| 19 | Methylenediaminetetraacetic dianhydride | 0.500% | 18.04 | 185.89 | 158.22 | 85.11% |
| 20 | Propylenediaminetetraacetic dianhydride | 0.005% | 17.34 | 187.02 | 156.84 | 83.86% |
| 21 | Propylenediaminetetraacetic dianhydride | 0.010% | 17.23 | 185.75 | 157.55 | 84.82% |
| 22 | Propylenediaminetetraacetic dianhydride | 0.030% | 17.4 | 184.18 | 158.32 | 85.96% |
| 23 | Propylenediaminetetraacetic dianhydride | 0.050% | 17.34 | 186.51 | 159.84 | 85.70% |
| 24 | Propylenediaminetetraacetic dianhydride | 0.100% | 17.33 | 185.19 | 157.98 | 85.31% |
| 25 | Propylenediaminetetraacetic dianhydride | 0.200% | 18.13 | 184.8 | 156.57 | 84.72% |
| 26 | Propylenediaminetetraacetic dianhydride | 0.300% | 18.27 | 185.34 | 155.99 | 84.16% |
| 27 | Propylenediaminetetraacetic dianhydride | 0.400% | 18.32 | 185.6 | 155.07 | 83.55% |
| 28 | Propylenediaminetetraacetic dianhydride | 0.500% | 18.01 | 184.28 | 153.78 | 83.45% |
| 29 | Butylenediaminetetraacetic dianhydride | 0.005% | 17.58 | 185.42 | 154.67 | 83.42% |
| 30 | Butylenediaminetetraacetic dianhydride | 0.010% | 17.43 | 185.21 | 156.54 | 84.52% |
| 31 | Butylenediaminetetraacetic dianhydride | 0.030% | 17.98 | 183.98 | 155.87 | 84.72% |
| 32 | Butylenediaminetetraacetic dianhydride | 0.050% | 17.73 | 185.62 | 155.76 | 83.91% |
| 33 | Butylenediaminetetraacetic dianhydride | 0.100% | 17.63 | 185.24 | 154.52 | 83.42% |
| 34 | Pentanediaminetetraacetic dianhydride | 0.005% | 18.02 | 185.31 | 154.67 | 83.47% |
| 35 | Pentanediaminetetraacetic dianhydride | 0.010% | 17.83 | 185.94 | 156.54 | 84.19% |
| 36 | Pentanediaminetetraacetic dianhydride | 0.030% | 17.71 | 184.65 | 155.87 | 84.41% |
| 37 | Pentanediaminetetraacetic dianhydride | 0.050% | 17.92 | 186.7 | 155.76 | 83.43% |
| 38 | Pentanediaminetetraacetic dianhydride | 0.100% | 18.65 | 185.48 | 153.52 | 82.77% |
| 39 | Hexamethylenediaminetetraacetic | 0.005% | 17.9 | 186.19 | 153.64 | 82.52% |
| | dianhydride | | | | | |
| 40 | Hexamethylenediaminetetraacetic | 0.010% | 17.95 | 185.93 | 153.15 | 82.37% |
| | dianhydride | | | | | |
| 41 | Hexamethylenediaminetetraacetic | 0.030% | 18.04 | 184.42 | 152.32 | 82.59% |
| | dianhydride | | | | | |
| 42 | Hexamethylenediaminetetraacetic | 0.050% | 18.57 | 186.66 | 150.87 | 80.83% |
| | dianhydride | | | | | |
| 43 | Hexamethylenediaminetetraacetic | 0.100% | 18.81 | 184.31 | 150.73 | 81.78% |
| | dianhydride | | | | | |
| 44 | 4,4′-(propane-1,2-diyl)bis(morpholine-2,6- | 0.005% | 17.44 | 186.43 | 157.64 | 84.56% |
| | dione) | | | | | |
| 45 | 4,4′-(propane-1,2-diyl)bis(morpholine-2,7- | 0.010% | 17.83 | 186.13 | 157.34 | 84.53% |
| | dione) | | | | | |
| 46 | 4,4′-(propane-1,2-diyl)bis(morpholine-2,8- | 0.030% | 17.08 | 185.85 | 158.48 | 85.27% |
| | dione) | | | | | |
| 47 | 4,4′-(propane-1,2-diyl)bis(morpholine-2,9- | 0.050% | 17 | 186.16 | 157.61 | 84.66% |
| | dione) | | | | | |
| 48 | 4,4′-(propane-1,2-diyl)bis(morpholine-2,10- | 0.100% | 16.76 | 184.03 | 157.83 | 85.76% |
| | dione) | | | | | |
| 49 | 4,4′-(propane-1,2-diyl)bis(morpholine-2,11- | 0.200% | 17.63 | 184.54 | 156.4 | 84.75% |
| | dione) | | | | | |
| 50 | 4,4′-(propane-1,2-diyl)bis(morpholine-2,12- | 0.300% | 17.76 | 185 | 158.19 | 85.51% |
| | dione) | | | | | |
| 51 | 4,4′-(propane-1,2-diyl)bis(morpholine-2,13- | 0.400% | 18.06 | 183.89 | 156.73 | 85.23% |
| | dione) | | | | | |
| 52 | 4,4′-(propane-1,2-diyl)bis(morpholine-2,14- | 0.500% | 18.77 | 184.89 | 154.82 | 83.74% |
| | dione) | | | | | |
| 53 | 4,4′-(butane-1,2-diyl)bis(morpholine-2,6- | 0.005% | 17.64 | 185.92 | 157.17 | 84.54% |
| | dione) | | | | | |
| 54 | 4,4′-(butane-1,2-diyl)bis(morpholine-2,7- | 0.010% | 17.58 | 186.44 | 156.47 | 83.93% |
| | dione) | | | | | |
| 55 | 4,4′-(butane-1,2-diyl)bis(morpholine-2,8- | 0.030% | 17.49 | 184.76 | 157.39 | 85.19% |
| | dione) | | | | | |
| 56 | 4,4′-(butane-1,2-diyl)bis(morpholine-2,9- | 0.050% | 17.48 | 186.34 | 156.44 | 83.95% |
| | dione) | | | | | |
| 57 | 4,4′-(butane-1,2-diyl)bis(morpholine-2,10- | 0.100% | 17.09 | 182.39 | 156.18 | 85.63% |
| | dione) | | | | | |
| 58 | 4,4′-(butane-1,2-diyl)bis(morpholine-2,11- | 0.200% | 18.17 | 184.63 | 156.36 | 84.69% |
| | dione) | | | | | |
| 59 | 4,4′-(butane-1,2-diyl)bis(morpholine-2,12- | 0.300% | 17.45 | 184.57 | 157.3 | 85.23% |
| | dione) | | | | | |
| 60 | 4,4′-(butane-1,2-diyl)bis(morpholine-2,13- | 0.400% | 18.55 | 183.75 | 155.04 | 84.38% |
| | dione) | | | | | |
| 61 | 4,4′-(butane-1,2-diyl)bis(morpholine-2,14- | 0.500% | 19.25 | 184.37 | 154.89 | 84.01% |
| | dione) | | | | | |
| 62 | 4,4′-(pentane-1,2-diyl)bis(morpholine-2,6- | 0.005% | 18.34 | 185.97 | 154.89 | 83.29% |
| | dione) | | | | | |
| 63 | 4,4′-(pentane-1,2-diyl)bis(morpholine-2,7- | 0.010% | 17.52 | 185.34 | 155.61 | 83.96% |
| | dione) | | | | | |
| 64 | 4,4′-(pentane-1,2-diyl)bis(morpholine-2,8- | 0.030% | 17.91 | 185.13 | 154.13 | 83.26% |
| | dione) | | | | | |
| 65 | 4,4′-(pentane-1,2-diyl)bis(morpholine-2,9- | 0.050% | 17.94 | 186.54 | 155.31 | 83.26% |
| | dione) | | | | | |
| 66 | 4,4′-(pentane-1,2-diyl)bis(morpholine-2,10- | 0.100% | 17.68 | 180.97 | 153.58 | 84.86% |
| | dione) | | | | | |
| 67 | 4,4′-(hexane-1,2-diyl)bis(morpholine-2,6- | 0.005% | 18.33 | 184.92 | 154.55 | 83.58% |
| | dione) | | | | | |
| 68 | 4,4′-(hexane-1,2-diyl)bis(morpholine-2,7- | 0.010% | 17.28 | 183.8 | 154.58 | 84.10% |
| | dione) | | | | | |
| 69 | 4,4′-(hexane-1,2-diyl)bis(morpholine-2,8- | 0.030% | 17.69 | 184.7 | 151.87 | 82.23% |
| | dione) | | | | | |
| 70 | 4,4′-(hexane-1,2-diyl)bis(morpholine-2,9- | 0.050% | 17.68 | 184.77 | 153.22 | 82.92% |
| | dione) | | | | | |
| 71 | 4,4′-(hexane-1,2-diyl)bis(morpholine-2,10- | 0.100% | 17.61 | 180.53 | 153.22 | 84.87% |
| | dione) | | | | | |
| 72 | 4,4′-(butane-2,3-diyl)bis(morpholine-2,6- | 0.005% | 17.43 | 185.27 | 156.6 | 84.53% |
| | dione) | | | | | |
| 73 | 4,4′-(butane-2,3-diyl)bis(morpholine-2,7- | 0.010% | 18.11 | 184.16 | 156.8 | 85.14% |
| | dione) | | | | | |
| 74 | 4,4′-(butane-2,3-diyl)bis(morpholine-2,8- | 0.030% | 17.25 | 183.56 | 157.1 | 85.59% |
| | dione) | | | | | |
| 75 | 4,4′-(butane-2,3-diyl)bis(morpholine-2,9- | 0.050% | 17.15 | 184.74 | 157.31 | 85.15% |
| | dione) | | | | | |
| 76 | 4,4′-(butane-2,3-diyl)bis(morpholine-2,10- | 0.100% | 16.74 | 183.35 | 157.96 | 86.15% |
| | dione) | | | | | |
| 77 | 4,4′-(butane-2,3-diyl)bis(morpholine-2,11- | 0.200% | 17.77 | 183.53 | 156.19 | 85.10% |
| | dione) | | | | | |
| 78 | 4,4′-(butane-2,3-diyl)bis(morpholine-2,12- | 0.300% | 17.91 | 184.55 | 157.02 | 85.08% |
| | dione) | | | | | |
| 79 | 4,4′-(butane-2,3-diyl)bis(morpholine-2,13- | 0.400% | 17.83 | 183.1 | 155.4 | 84.87% |
| | dione) | | | | | |
| 80 | 4,4′-(butane-2,3-diyl)bis(morpholine-2,14- | 0.500% | 19.47 | 185.24 | 155.39 | 83.89% |
| | dione) | | | | | |
| 81 | 4,4′-(hexane-3,4-diyl)bis(morpholine-2,6- | 0.005% | 18.93 | 184.6 | 151.27 | 81.94% |
| | dione) | | | | | |
| 82 | 4,4′-(hexane-3,4-diyl)bis(morpholine-2,7- | 0.010% | 17.72 | 184.82 | 152.58 | 82.56% |
| | dione) | | | | | |
| 83 | 4,4′-(hexane-3,4-diyl)bis(morpholine-2,8- | 0.030% | 18.28 | 183.12 | 152.82 | 83.45% |
| | dione) | | | | | |
| 84 | 4,4′-(hexane-3,4-diyl)bis(morpholine-2,9- | 0.050% | 17.77 | 184.98 | 150.01 | 81.10% |
| | dione) | | | | | |
| 85 | 4,4′-(hexane-3,4-diyl)bis(morpholine-2,10- | 0.100% | 17.45 | 181.02 | 152.17 | 84.06% |
| | dione) | | | | | |

From the description above, it can be determined that the examples of the present disclosure achieve the following technical effects:

By comparing Examples 1-85 with Comparative Example 1, and referring to FIGS. 1-3, it can be determined that the electrolyte additive of the present application can improve the cycle performance and/or reduce the cycle impedance of the lithium ion secondary battery. It should be understood that, in combination with actually used products, batteries in different usage scenarios may have different performance requirements: a long-cycle battery requires a longer cycle life, and therefore a slightly higher impedance may be acceptable as long as the effect on the cycle is not obvious; and a power battery may have higher requirement for impedance, and a slightly lower cycle life is also acceptable. Therefore, even if in some cases, the additive of the present application only achieves improvement in one of the cycle performance and the cycle impedance with respect to a battery without the additive, it is still advantageous in specific battery applications.

By comparing Examples 1-28, 44-61 and 72-80 with Comparative Example 1, and referring to FIGS. 1-3, it can be determined that when the electrolyte additive of the present application is methylenediaminetetraacetic dianhydride, ethylenediaminetetraacetic dianhydride, propylenediaminetetraacetic dianhydride, 4,4′-(propane-1,2-diyl)bis(morpholine-2,6-dione), 4,4′-(butane-1,2-diyl)bis(morpholine-2,6-dione), and 4,4′-(butane-2,3-diyl)bis(morpholine-2,6-dione), the improvement on the cycle performance and cycle impedance of the lithium ion secondary battery is more significant.

By comparing Examples 1-10, it can be determined that based on the total mass of the electrolyte, when the amount of the additive of the present application is 0.005 wt %-0.5 wt %, it is beneficial to further improve the cycle performance and cycle impedance of the lithium ion secondary battery.

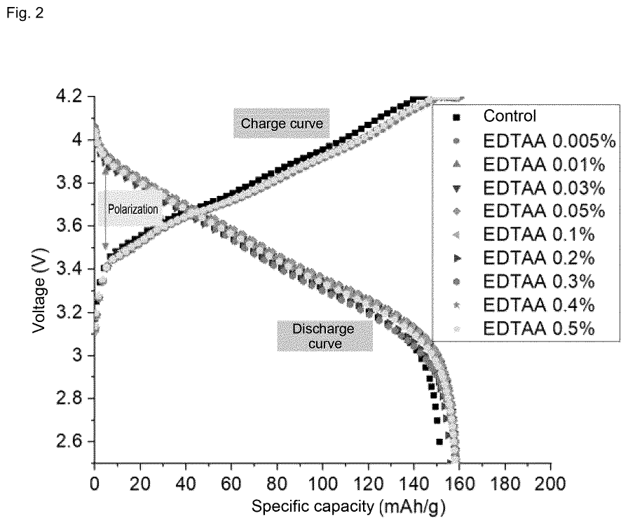

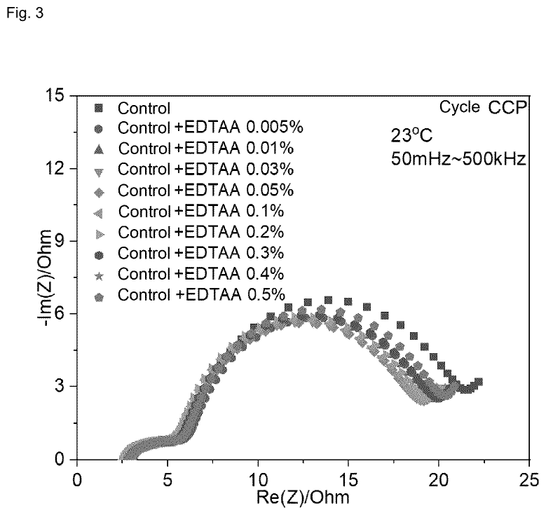

In addition, it can be determined from FIGS. 2 and 3 that compared with Comparative Example 1, the batteries of Examples 1-9 have a comparable or better capacity, a shorter constant-voltage plateau, and a lower cycle impedance, and thus better performance.

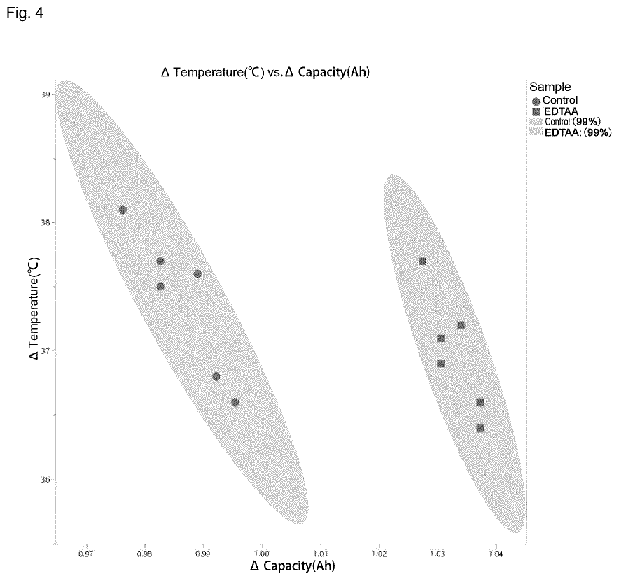

In addition, the electrolytes of Example 9 and Comparative Example 1 were used to assemble into 1 Ah-class pouch batteries, and the batteries were subjected to a charge and discharge test (the test conditions were 23 °C, 11 A discharge, and a cut-off voltage of 2.5V). During the test, the battery was thermally insulated, a temperature measurement thermocouple was attached to the battery to measure temperature data, and capacity data was synchronously obtained by means of a charging and discharging device. Three batteries were assembled using each of the electrolytes of Example 9 and Comparative Example 1, and each battery was tested twice to obtain the graph of capacity and temperature variations in a battery cycle of FIG. 4. The graph shows comparison of battery capacity and temperature rise under a large current, where A capacity in the graph is a capacity discharged during battery discharging, and A temperature is temperature variation during battery discharging. 99% in the legend refers to a possible range of data coverage in the graph.

It can be determined from FIG. 4 that during large-rate discharge in the 1 Ah-class batteries, the battery capacity of Example 9 is greater than that of Comparative Example 1, and the temperature rise is less than that of Comparative Example 1, which proves that the electrolyte additive of the present application can improve the cycle performance and reduce the cycle impedance of the battery.

It is to be noted that terms “first”, “second” and the like in the description and claims of the present application are used for distinguishing similar objects rather than describing a specific sequence or a precedence order. It should be understood that the terms so used may be interchanged where appropriate so that the embodiments of the present application described herein can be implemented in sequences other than those described herein.

It should be understood that various changes and modifications to the embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. An additive for an electrolyte of a lithium ion battery, the additive comprising a compound having a structure of formula (1) 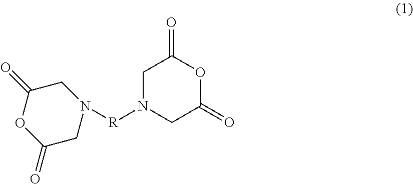
wherein R is a substituted or unsubstituted linear or branched C1-6 hydrocarbon group.

2. The additive according to claim 1, wherein in formula (1), R is an unsubstituted linear or branched C1-6 alkyl group.

3. The additive according to claim 1, wherein the compound having the structure of formula (1) is methylenediaminetetraacetic dianhydride, ethylenediaminetetraacetic dianhydride, propylenediaminetetraacetic dianhydride, butylenediaminetetraacetic dianhydride, pentanediaminetetraacetic dianhydride, hexamethylenediaminetetraacetic dianhydride, 4,4′-(propane-1,2-diyl)bis(morpholine-2,6-dione), 4,4′-(butane-1,2-diyl)bis(morpholine-2,6-dione), 4,4′-(pentane-1,2-diyl)bis(morpholine-2,6-dione), 4,4′-(hexane-1,2-diyl)bis(morpholine-2,6-dione), 4,4′-(butane-2,3-diyl)bis(morpholine-2,6-dione) or 4,4′-(hexane-3,4-diyl)bis(morpholine-2,6-dione).

4. An electrolyte for a lithium ion battery, wherein the electrolyte comprises the additive according to claim 1, an organic solvent, and a lithium salt.

5. The electrolyte according to claim 4, wherein based on a total mass of the electrolyte, an amount of the additive is 0.005 wt %-0.5 wt %.

6. The electrolyte according to claim 4, wherein the organic solvent comprises a cyclic carbonate, a linear carbonate, or any combination thereof.

7. The electrolyte according to claim 4, wherein the lithium salt is selected from a group consisting of lithium hexafluorophosphate, lithium bis(trifluoromethylsulfonyl)imide, lithium trifluoromethylsulfonate, lithium bis(fluorosulfonyl)imide, lithium tetrafluoroborate, lithium bis(oxalate) borate, lithium difluorooxalate borate, lithium perchlorate, or a combination thereof.

8. A lithium ion secondary battery, comprising:
a positive electrode plate,
a negative electrode plate,
a separator, and
the electrolyte according to claim 4.

9. The lithium ion secondary battery according to claim 8, wherein the positive electrode plate comprises a ternary or lithium cobalt oxide positive electrode material.

10. The lithium ion secondary battery according to claim 8, wherein the negative electrode plate comprises a graphite negative electrode material or a silicon-carbon negative electrode material.
